(19) United States
(12) Patent Application Publication
(10) Pub. No.: US 2021/0269770 A1
(43) Pub. Date: Sep. 2, 2021

(54) EXPANSION AND DIFFERENTIATION OF NEURONAL PRECURSOR CELLS

(71) Applicant: SKIN2NEURON PTY LTD, Sydney (AU)

(72) Inventors: Michael VALENZUELA, Sydney (AU); Tom DUNCAN, Sydney (AU); An TRUONG, Sydney (AU)

(21) Appl. No.: 17/254,232

(22) PCT Filed: Jun. 21, 2019
(86) PCT No.: PCT/AU2019/050637

§ 371 Date: Dec. 18, 2020

(30) Foreign Application Priority Data
Jun. 22, 2018 (AU) 2018902237

(51) Int. Cl. C12N 5/0793

(52) U.S. Cl. CPC C12N 5/0619; C12N 2506/092; C12N 2501/155; C12N 2501/40; C12N 2509/10; C12N 2501/15; C12N 2500/84; C12N 2509/00; C12N 2501/41

(57) ABSTRACT

The invention relates to preparation of neuronal precursor cells, compositions comprising same and therapeutic uses.

### CROSS-REFERENCE TO RELATED APPLICATIONS

This application is the national stage of International Patent Application No. PCT/AU2019/050637, filed Jun. 21, 2019, which claims the benefit of priority from Australian Patent Application No. 2018902237, filed Jun. 22, 2018, which are incorporated herein by reference in their entirety.

### FIELD OF THE INVENTION

The invention relates to preparation of neuronal precursor cells, compositions comprising same and therapeutic uses.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Multipotent stem cells are the customary starting point for manufacturing neurons de novo—expansion of cells in their primitive replicative state is followed by directed differentiation into a mature neuronal phenotype. Multipotent stem cells can be isolated from embryonic origins (i.e., embryonic stem cells) or adult stem cell reservoirs (i.e., adult stem cells such as mesenchymal stem cells), or as more recently discovered, by reprogramming of mature post-differentiated cells (e.g, fibroblasts) into embryonic-like stem cells (i.e., induced pluripotential stem cells).

Each of these methods have a general limitation, by definition, of being capable of multiple cell fates (i.e., multipotent). Neuronal yields are therefore low and variable, resulting in non-neuronal cell phenotypes even after treatment with specific neuronal differentiation conditions. Glial cell differentiation after such treatment is, for example, a common limitation. For the purpose of biological research, commercial application or therapeutic use it would be useful to be able to produce a homogenous population of unipotent neural precursors, that is, cells that are uniform, transiently replicative and fate-committed to neuronal lineages.

The closest approximation to date has been direct genetic reprogramming of mature post-differentiated cells (e.g., fibroblasts) into post mitotic neurons, by-passing the proliferative stem cell or precursor stage through upregulation of key neuronal induction genes (Vierbuchen et al., 2010, Pang et al., 2011, Son et al., 2011, Zhou et al., 2014, Tsunemoto et al., 2018). However, this method relies on genetic manipulation and does not produce an expandable population, and like all methods alluded to suffers from unacceptably high line-to-line variability (Truong et al., 2016).

With this in mind it is interesting that human skin and the central nervous system share the same embryologic origins, the ectoderm germ lineage. It is rarely appreciated that adult stem cells and precursors in these two organs express many of the same cell markers (e.g., nestin, CD133, Sox2, etc) and utilize many of the same cell cycle regulatory factors (Noggin, SHH, FGF, EGF, BDNF, etc).

In 2001, Toma et al. 2001) demonstrated for the first time that multipotent stem cells in the mammalian hair follicle niche could indeed produce neurons in vitro, albeit at low yield after differentiation (<15%), with most cells developing into non-neuronal cell types. This basic result has since been replicated using human skin by this group (Vierbuchen et al., 2010, Pang et al., 2011, Son et al., 2011, Zhou et al., 2014, Tsunemoto et al., 2018) and others (Joannides et al., 2004, Yu et al., 2010, Yu et al., 2006, Belicchi et al., 2004). Interestingly, the converse has also recently been shown: Hwang et al. (2016) found that bona fide neural stem cell extracts (of human foetal origin) can increase hair regrowth in vivo in depilated mice.

Hence, whilst both organ systems differ vastly in structure and function, they conserve much of the same biological machinery governing stem and precursor cell proliferation. Furthermore, stem cells and precursors from one niche can assume the role and identity of the other by virtue of environmental cues alone. Given the difficulty in accessing adult human neural stem cells from their physiological niche (in the brain), it is conceptually appealing that neural precursors could be derived for a given individual from their own hair follicle precursor population without resorting to genetic manipulation.

To date, this premise has failed in practice. Cell viability and neuronal yields from native human skin have been too low and line-to-line variability too great.

The source of precursors in this context—the adult human hair follicle—contains both unipotent precursor cells as well as multipotent stem cells. Unipotent precursors are fundamentally different from multipotent stem cells by virtue of fate restriction to only one cell lineage and being incapable of indefinite cell replication in vitro. Recently, it has emerged that there is great heterogeneity amongst transiently amplifying precursor cells in the hair follicle, and this variation allows for development of subpopulations with different lineage fate (Yang et al., 2017). Furthermore, this “lineage infidelity” emerges maximally under conditions of wound repair, tumorigenic transformation or in vitro cell culture (Ge et al., 2017, Fuchs, 2018).

Hitherto unrecognized has been the ability of a sub-population of human hair follicle stem or precursor cells to harbor a latent neuronal fate restriction.

Facility to isolate and expand such unipotent neural precursors from mature skin diverges widely between species. For example, we have previously developed a process on adult canine skin (Valenzuela et al. (2008), but this method is not generally applicable to human skin because of large inter- and intra-individual differences in hair follicle quantity and quality.

### SUMMARY OF THE INVENTION

In one embodiment there is provided a method for producing a composition of neuronal precursor cells, or of cells capable of proliferation that express neural lineage biomarkers including:
treating a sample of hair follicle cells in conditions enabling the transition of hair follicle cells to a growth phase, thereby forming a sample of conditioned cells;
treating the sample of conditioned cells in conditions enabling enrichment of the number of cells in the sample that contain neuronal lineage biomarkers;

thereby producing the composition of neuronal precursor cell, or of cells capable of proliferation that express neural lineage biomarkers.

In one embodiment there is provided a method for producing a composition of neuronal precursor cells, or of cells capable of proliferation that express neural lineage biomarkers including:
treating a sample of hair follicle cells in conditions enabling the transition of hair follicle cells to a growth phase, thereby forming a sample of conditioned cells;
treating the sample of conditioned cells in conditions enabling the formation of neurospheres from the cells of the sample of conditioned cells;

thereby producing the composition of neuronal precursor cells, or of cells capable of proliferation that express neural lineage biomarkers.

In one embodiment there is provided a method for producing a composition of neuronal precursor cells, or of cells capable of proliferation that express neural lineage biomarkers including:
treating a sample of hair follicle cells in conditions enabling the transition of hair follicle cells to a growth phase, thereby forming a sample of conditioned cells;
treating the sample of conditioned cells in conditions enabling the formation of neurospheres from the cells of the sample of conditioned cells;
providing conditions to the sample of neurospheres to expand the number of cells of the neurospheres;

thereby producing the composition of neuronal precursor cells, or of cells capable of proliferation that express neural lineage biomarkers.

In one embodiment there is provided a method for producing a composition of neuronal precursor cells, or of cells capable of proliferation that express neural lineage biomarkers including:
treating a sample of hair follicle cells in conditions enabling the transition of hair follicle cells to a growth phase, thereby forming a sample of conditioned cells;
providing conditions to the sample of conditioned cells to enable dissociation of cells into single cells;
treating the sample of conditioned cells in conditions enabling the formation of neurospheres from the cells of the sample of conditioned cells;
providing conditions to the sample of neurospheres to expand the number of cells of the neurospheres;

thereby producing the composition of neuronal precursor cells, or of cells capable of proliferation that express neural lineage biomarkers.

In one embodiment there is provided a method for producing a composition of neuronal precursor cells, or of cells capable of proliferation that express neural lineage biomarkers including:
treating a sample of skin, the skin including hair follicle cells, in conditions enabling the transition of hair follicle cells in the skin to a growth phase, thereby forming a sample of conditioned skin;
treating the sample of conditioned skin in conditions enabling enrichment of the number of cells in the sample that contain neuronal lineage biomarkers;

thereby producing the composition of neuronal precursor cells, or of cells capable of proliferation that express neural lineage biomarkers.

In one embodiment there is provided a method for producing a composition of neuronal precursor cells, or of cells capable of proliferation that express neural lineage biomarkers including:
treating a sample of skin, the skin including hair follicle cells, in conditions enabling the transition of hair follicle cells in the skin to a growth phase, thereby forming a sample of conditioned skin;
depleting terminally differentiated cells or apoptotic cells or cell debris from the sample of conditioned skin, thereby forming a sample of non-terminally differentiated cells;
treating the sample of non-terminally differentiated cells in conditions enabling enrichment of the number of cells in the sample that contain neuronal lineage biomarkers;

thereby producing the composition of neuronal precursor cells, or of cells capable of proliferation that express neural lineage biomarkers.

In one embodiment there is provided a method for producing a composition of neuronal precursor cells, or of cells capable of proliferation that express neural lineage biomarkers including:
treating a sample of skin, the skin including hair follicle cells, in conditions enabling the transition of hair follicle cells in the skin to a growth phase, thereby forming a sample of conditioned skin;
releasing cells from the conditioned skin into the sample;
depleting terminally differentiated cells or apoptotic cells or cell debris from the sample of conditioned skin, thereby forming a sample of non-terminally differentiated cells;
treating the sample of non-terminally differentiated cells in conditions enabling enrichment of the number of cells in the sample that contain neuronal lineage biomarkers;

thereby producing the composition of neuronal precursor cells, or of cells capable of proliferation that express neural lineage biomarkers.

In further embodiments there is provided a composition of cells produced by the above described method. The composition may comprise a therapeutically effective amount of cells and a pharmaceutically acceptable diluent, carrier or excipient. The cellular component of the composition may consist of cells produced by a method described above, or the cellular component may comprise cells produced by a method described above and further comprise other cells. The composition may be adapted to enable injection or infusion.

In further embodiments there is provided a use of the composition of cells for therapy, for example for therapy of a condition or disease of neural tissue. Thus in one embodiment there is provided a method for treatment of a disease or condition, preferably a disease or condition of neural tissue, in an individual requiring said treatment comprising administering a composition described above to an individual requiring said treatment, thereby treating said disease or condition in said individual.

In further embodiments there is provided a composition of cells described above for use in treatment of a disease or condition, preferably a disease or condition of neural tissue.

In further embodiments there is provided a use of a composition of cells described above in the manufacture of a medicament for treatment of a disease or condition, preferably a disease or condition of neural tissue.

In a further embodiment there is provided one or more devices for treatment of a disease or condition, preferably a disease or condition of neural tissue comprising a composition of cells described above.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example.

### BRIEF DESCRIPTION OF THE DRAWINGS

FIG. 1. Typical cell yield per hair follicle after neurosphere dissociation with and without prior chemical pretreatment with sonic hedge hog (SHH) for 6 or 72 hours. Data from a 45-year old male.

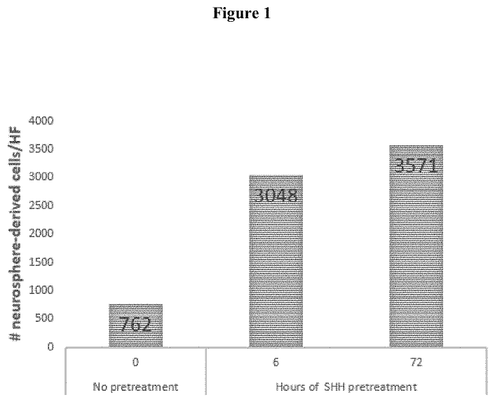

FIG. 2. Superior cellular yield following immediate hair follicle enzymatic treatment with collagenase/dispase treatment and trituration.

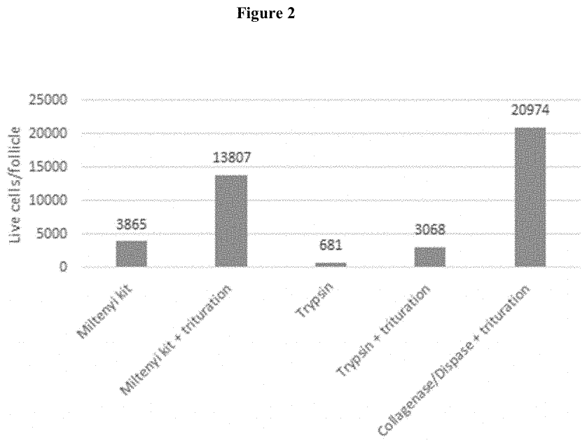

FIG. 3. Brightfield micrographs of typical neurospheres derived from hair follicles of a 45-year old male (top row) and 25-year old male (bottom row).

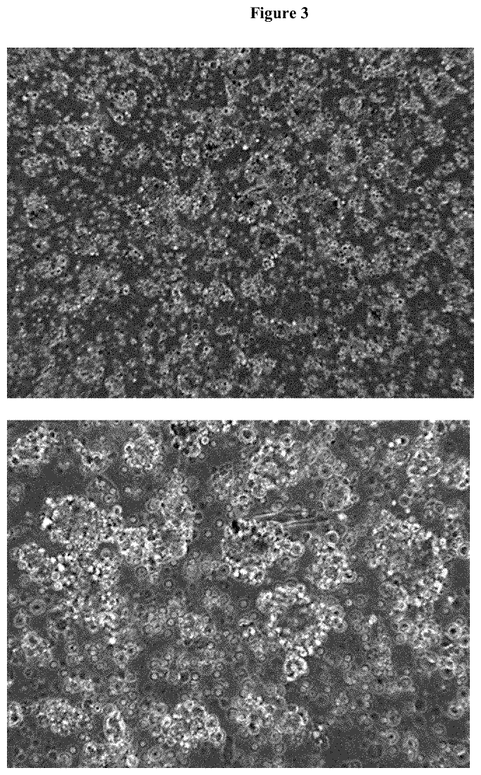

FIG. 4. Fluorescent micrographs of neurospheres stained for a range of typical neural precursor biomarkers. A) Ubiquitous neural stem cell marker NESTIN (>90% of cells positive). B) neural progenitor marker DOUBLECORTIN (DCX; >80% of cells positive). C) immature neuronal marker βIII-TUBULIN (B3T; >50% of cells positive). D) negative control (no primary). All neurospheres derived from hair follicles of a 34-year old male.

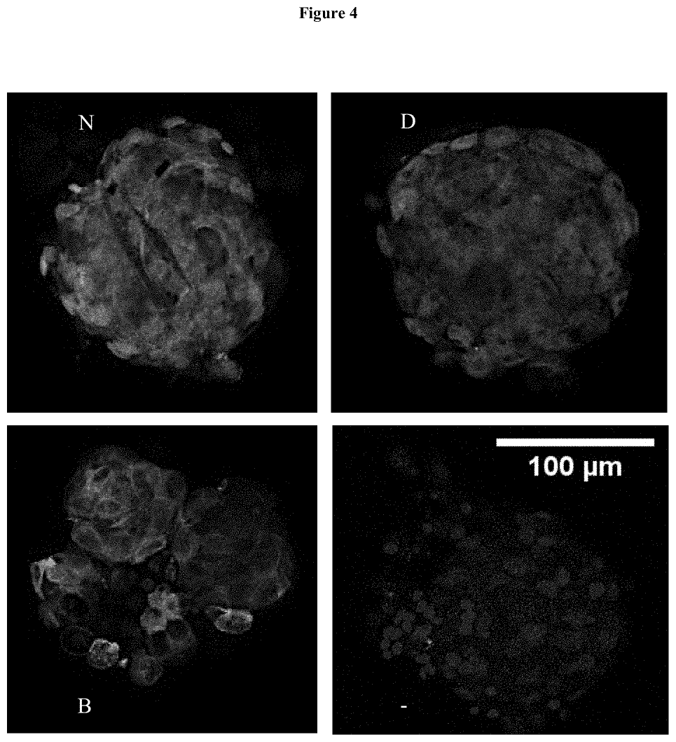

FIG. 5. Monolayer expansion of HFNs. Brightfield micrograph of typical morphology of confluent HFNs from 25-year old male. Fluorescence images showing ubiquitous (>90% positive) protein expression of stem cell marker CD133 and neural stem cell marker NESTIN and immature neuronal marker βIII-TUBULIN (B3T). Fluorescent images of HFN cells from a 45 yo male.

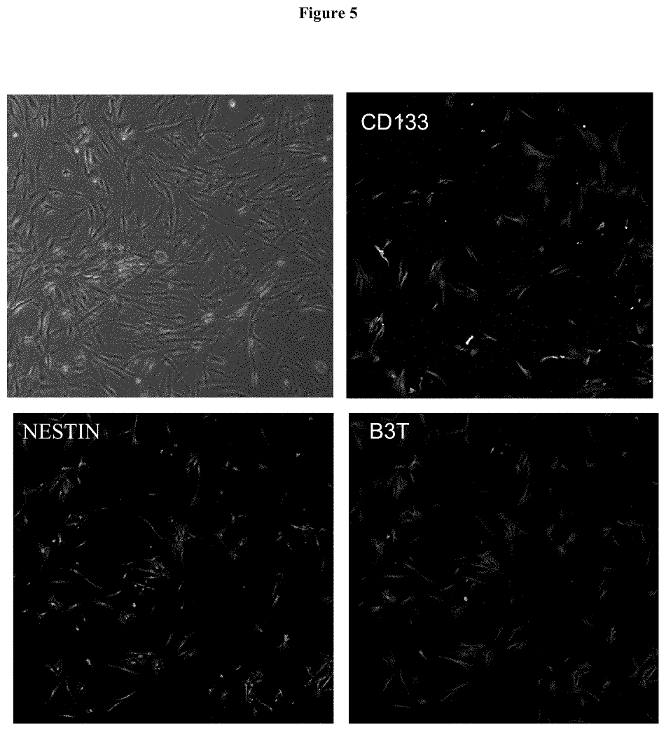

### DETAILED DESCRIPTION OF THE EMBODIMENTS

The invention provides a method for producing a precursor neuronal cell composition that enables the production of genetically unmodified human cells that show limited line to line variability and capacity to rapidly expand into commercially useful cell numbers. The method includes:
treating a sample of hair follicle cells in conditions enabling the transition of hair follicle cells to a growth phase, thereby forming a sample of conditioned cells;
treating the sample of conditioned cells in conditions enabling enrichment of the number of cells that contain neuronal lineage biomarkers in the sample;

thereby producing the composition of neuronal precursor cells or cells capable of proliferation that express neural lineage biomarkers.

The cells of the composition produced by the method are neuronal precursor cells or more generally proliferating cells that express neural lineage biomarkers. Neural lineage biomarkers cover a developmental spectrum that can include (most primitively) neural stem cell-like marker nestin, radial glial cell marker GFAP, neuroblastic markers doublecortin (DCX) and NCAM and immature neuron marker betaIII-tubulin. More generic stem cell-like markers such as CD133, Sox2 and OCT4 can also be expressed. Cells positive for these markers are enriched in neurospheres and that have potential to form terminally differentiated neurons. Generally, these cells are multipotent, that is having potency for the generation of terminally differentiated cells of the neural lineage, such as neurons and glia.

Generally an initial step of the method involves the treatment of hair follicle cells. As explained below, these cells may be obtained in the form of a sample of skin having hair follicles. Alternatively, these cells may be obtained from a hair follicle isolate. As is generally known, hair follicle cells may exist in a range of phases (anagen, catagen and telogen) in the context of growth. Telogen is a resting phase (i.e. no growth), anagen is a growth phase and catagen is an intermediate phase between anagen and telogen. The treatment step results in the enrichment of hair follicle cells that are in a growth phase or anagen phase. The enrichment may arise from stimulation of hair follicle cells so that those cells in telogen phase transition to anagen phase, and/or from preventing cells in anagen phase from transitioning to catagen phase. There is a particular enrichment of hair follicle cells that possess potential for development of neural lineage biomarkers in the anagen phase as a result of the treatment step.

In a further step, the cells are treated to enrich, or to otherwise, increase the number of cells containing neuronal lineage biomarkers. This can be achieved by a treatment that increases the number of cells that contain neuronal lineage biomarkers and/or by decreasing the number of cells that do not contain neuronal lineage biomarkers. In a particularly preferred step, the cells are treated in conditions enabling the formation of neurospheres. Methods for neurosphere formation are generally well known in the art and exemplified further herein. In one embodiment, the cells are treated to enrich or otherwise to increase the number of cells containing neuronal lineage biomarkers, or to enable the formation of neurospheres by culturing the cells in a bioreactor. A bioreactor may be utilised to provide improved conditions for formation of neurospheres.

A first embodiment of the invention is now described in which a sample of skin including hair follicles is utilised to derive a composition of neuronal precursor cells, according to which a sample of skin that includes hair follicle cells is treated in conditions enabling the transition of hair follicle cells in the skin to a growth phase, thereby forming a sample of conditioned skin; and thereafter, the sample of conditioned cells is treated in conditions enabling enrichment of the number of cells containing neural lineage specific biomarkers in the sample, to thereby produce a composition of neuronal precursor cells.

A first step of the method may involve the harvesting of a skin sample that contains hair follicle cells. It is preferred that the sample is obtained from human scalp skin that contains the highest and most uniform hair follicle density, preferably midline occipital scalp skin. This skin generally contains a higher density of active hair follicles from individual to individual. We have found that a skin region containing a higher density of active hair follicles enables the method to produce a greater quantity and quality of cells, as compared with skin regions that contain a lower density of hair follicles.

The cells of the hair follicles generally include precursor and stem cells from different niches, including the bulge area, the germ zone and the dermal papillae, collectively referred to as hair follicular precursors. Hair follicular precursors can express neuro-ectodermal biomarkers.

The harvested skin sample is then treated in conditions enabling substantially all of the hair follicular precursors to transition to a growth phase. The step is important because it contributes to increased yield of neural precursor cells. An exemplification of the step is described in some detail in the examples herein.

The step generally involves the in vitro conditioning of the human skin sample, the purpose of which is to provide conditions that support cell survival of cells of the skin sample and enable the transition of cells to an anagen phase (or active growth phase of the follicle cell cycle).

Prior to this step, the cells of the skin sample may be in anagen, telogen or any of the other phases of the follicle cycle. It is preferred that the majority of hair follicle precursors are in the anagen phase at the time of harvest, although this is not necessary, because the end result of the in vitro culture is to transition the majority of hair follicular precursors to anagen phase.

Anti-refractory hair follicle factors and/or pro-growth factors may be utilised for promoting transition of hair follicle precursor and stem cells to anagen phase, thereby enabling the transition of hair follicle cells in the skin to a growth phase. Examples of anti-refractory hair follicle factors include noggin or sonic hedgehog (SHH) or factors that activate that Wnt1 signalling pathway, or that inhibit the bone morphogenic protein (pro-refractory) stimulus. Other examples of anti-refractory factors include WNTs, as well as BMP antagonists such as Grem 1 and Bambi. TGF-□2 is a key pro-growth factor, other examples of pro-growth factors include FGF7, FGF10 and platelet-derived growth factor (PDGF).

The in vitro conditioning of the sample of skin generally utilises a cell culture medium that supports the in vitro viability of epithelial cells and hair follicular organogenesis. Williams medium E is one example. Other examples include Dulbecco's modified eagle medium (DMEM) plus F-12 in different combinations, F12 plus mammalian serum in different combinations and different supplemented phosphate buffered saline combinations.

Generally the in vitro conditioning is for a period from 12 to 100 hours, although in some circumstances, it may be possible to culture for a longer period of time.

It is possible to monitor the progress of the in vitro culture during the culture period. For example, one may assess cell or culture characteristics by the growth of hair shafts within isolated hair follicles.

It is preferred that at completion of the in vitro cell culture, at least 80% of cells are in an anagen phase.

At the completion of the culture whereby a sample of conditioned skin is obtained, many of the cells will remain entrapped within the conditioned skin by the extra cellular matrix and non-cellular components of the skin tissue. These cells are to be released from the skin tissue. According to the method, the cells are released from the conditioned skin by contacting the skin with one or more enzymes in conditions enabling degradation of the extracellular matrix of the conditioned skin for release of the cells into the sample. Generally, the enzyme is one or more selected from the group consisting of trypsin, DNase, dispase, collagenase, and combinations of Accustase and TrypLE.

Depending on the nature of the conditioned skin, it may alternatively be possible to release cells from the conditioned skin by mechanical means that mince or separate tissue into smaller particles by disrupting the conditioned tissue.

In some embodiments, the cells are released from the conditioned skin by a combination of enzymatic and mechanical treatment. The enzymatic and mechanical treatment may occur at the same time, although generally the enzymatic treatment is initiated before the mechanical treatment is initiated.

At the completion of the step of releasing cells from the conditioned skin tissue sample there is provided released cells that may be suspended in cell medium and non cellular components of the extra cellular matrix and other non cellular components of skin tissue. These are generally removed before the initiation of subsequent steps. Centrifugation and filtration may be utilised. An exemplification of the approach is described in the Examples herein.

The composition of cells released from the conditioned skin is heterogeneous, including neuronal precursor cells, other multipotent cells, and terminally differentiated cells including fibroblasts, keratinocytes and other cells of the epidermal and dermal layers of skin tissue, depending on from where the tissue was harvested. The next step requires the obtaining of a sample of cells that is predominantly comprised of neural precursor cells. This then requires the removal of cells that are non-neural precursor cells from the composition of cells released from the conditioned human skin. The cells to be removed are typically terminally differentiated cells (i.e. keratinocytes, fibroblasts, other dermal and epidermal cells and apoptotic cells.

In one embodiment, the terminally differentiated cells are depleted from the sample by contacting the sample with a reagent for selectively depleting terminally differentiated cells from the sample in conditions enabling selective depletion of terminally differentiated cells from the sample. Preferably the agent is an antibody that binds to terminally differentiated cells but not to non-terminally differentiated cells. Antibody, preferably a monoclonal antibody that does not bind to neural precursor cells, is effective for this step. Preferably the antibody binds to cells of the epidermis or dermis such as keratinocytes, or to fibroblasts. Examples of antibody include those that selectively bind to fibroblast-specific antigen 1, or to CD45, these not being antigens found on the surface of neuronal precursor cells.

At the completion of the depletion step, the sample contains no more than about 5% by number of terminally differentiated cells.

Thus, in accordance with the first embodiment of the invention there is provided a method for producing a composition of neuronal precursor cells, or of cells capable of proliferation that express neural lineage biomarkers including:
treating a sample of skin, the skin including hair follicle cells, in conditions enabling the transition of hair follicle cells in the skin to a growth phase, thereby forming a sample of conditioned skin;
releasing cells from the conditioned skin into the sample;
depleting terminally differentiated cells or apoptotic cells or cell debris from the sample, thereby forming a sample of non-terminally differentiated cells; and optionally
treating the sample of non-terminally differentiated cells in conditions enabling expansion of the number of non-terminally differentiated cells in the sample;

thereby producing the composition of neuronal precursor cells.

The method may be implemented without a separate step of releasing cells from the conditioned skin. For example, cells may be released from the skin into the sample in the conditions of the treatment step by which hair follicle cells transition to a growth phase. Thus the method may broadly include producing a composition that is enriched for neuronal precursor cells, or cells capable of proliferation that express neural lineage biomarkers by:
treating a sample of skin, the skin including hair follicle cells, in conditions enabling the transition of hair follicle cells in the skin to a growth phase, thereby forming a sample of conditioned skin;
depleting terminally differentiated cells or apoptotic cells or cell debris from the sample of conditioned skin, thereby forming a sample of non-terminally differentiated cells;
treating the sample of non-terminally differentiated cells in conditions enabling enrichment of the number of cells containing neuronal lineage biomarkers in the sample;

thereby producing the composition of neuronal precursor cells or cells capable of proliferation that express neural lineage biomarkers.

Otherwise, it may be necessary to implement a separate process step for releasing cells from the condition skin as in a method including:
treating a sample of skin, the skin including hair follicle cells, in conditions enabling the transition of hair follicle cells in the skin to a growth phase, thereby forming a sample of conditioned skin;
releasing cells from the conditioned skin into the sample;
depleting terminally differentiated cells or apoptotic cells or cell debris from the sample of conditioned skin, thereby forming a sample of non-terminally differentiated cells;
treating the sample of non-terminally differentiated cells in conditions enabling enrichment of the number of cells containing neuronal lineage biomarkers in the sample;

thereby producing the composition of neuronal precursor cells or cells capable of proliferation that express neural lineage biomarkers.

In some embodiments, the step of depleting terminally differentiated cells from the sample of conditioned skin may be undertaken during, or as part of the step of—treating the sample of non-terminally differentiated cells in conditions enabling enrichment of the number of cells containing neuronal lineage biomarkers. For example, in one embodiment the conditions that result in an enrichment of the number of cells containing neuronal lineage biomarkers may include conditions that favour the loss of terminally differentiated cells, multipotent cells, or cells having potency for other than the generation of cells of the neural lineage. Thus in one embodiment the method may include:
treating a sample of skin, the skin including hair follicle cells, in conditions enabling the transition of hair follicle cells in the skin to a growth phase, thereby forming a sample of conditioned skin;
optionally, releasing cells from the conditioned skin into the sample;
treating the sample of cells from the conditioned skin in conditions enabling enrichment of the number of cells containing neuronal lineage biomarkers in the sample;

thereby producing the composition of neuronal precursor cells or cells capable of proliferation that express neural lineage biomarkers.

A second embodiment of the invention is now described in which a sample of hair follicles that are isolated from, or otherwise separated from so as not to be attached to, the skin tissue that attaches to them in the native state is utilised to derive a composition of neuronal precursor cells. One particular advantage of the use of isolated hair follicles is that the method can be implemented without the substantial use of enzymes or mechanical means that are otherwise required for the release of hair follicle cells from surrounding skin tissue. Further this also avoids the contamination of subsequent culture with terminally differentiated dermal cells and non neuronal lineage cells. According to the embodiment, the method produces a composition that is enriched for neuronal precursor cells, or cells capable of proliferation that express neural lineage biomarkers and includes the following steps:
treating a sample of hair follicle cells in conditions enabling the transition of hair follicle cells to a growth phase, thereby forming a sample of conditioned cells;
treating the sample of conditioned cells in conditions enabling the formation of neurospheres from the cells of the sample of conditioned cells;

thereby producing the composition of neuronal precursor cells or cells capable of proliferation that express neural lineage biomarkers.

The sample of conditioned cells is generally enriched for hair follicle cells in a growth phase and contains cells displaying neuronal lineage biomarkers.

At the completion of the formation of neurospheres, it may be advantageous to increase the number of cells of the neurospheres that are neural precursor cells, or that express neural lineage biomarkers, by an expansion step. There are a variety of techniques known for this purpose. Thus in another embodiment there is provided a method for producing a composition that is enriched for neuronal precursor cells, or cells capable of proliferation that express neural lineage biomarkers including:
treating a sample of hair follicle cells in conditions enabling the transition of hair follicle cells to a growth phase, thereby forming a sample of conditioned cells;
treating the sample of conditioned cells in conditions enabling the formation of a sample of neurospheres from the cells of the sample of conditioned cells;
providing conditions to the sample of neurospheres to expand the number of cells of the neurospheres;

thereby producing the composition of neuronal precursor cells or cells capable of proliferation that express neural lineage biomarkers.

At the completion of the step of treating hair follicle cells to transition cells to a growth phase, the cells may be clumped or grouped with other cells or materials, in which case the cells may be dissociated to form a suspension of single cells. This can be achieved by a number of techniques known in the art. In one example, enzymatic dissociation using enzymes that are useful for dissociating hair follicle cells to single cells is used. Thus in another embodiment there is provided a method for producing a composition that is enriched for neuronal precursor cells, or cells capable of proliferation that express neural lineage biomarkers including:
treating a sample of hair follicle cells in conditions enabling the transition of hair follicle cells to a growth phase, thereby forming a sample of conditioned cells;
providing conditions to the sample of conditioned cells to enable dissociation of cells into single cells;
treating the sample of conditioned cells in conditions enabling the formation of a sample of neurospheres from the cells of the sample of conditioned cells;
providing conditions to the sample of neurospheres to expand the number of cells of the neurospheres;

thereby producing the composition of neuronal precursor cells or cells capable of proliferation that express neural lineage biomarkers.

According to the second embodiment of the invention, a sample of about 200 hair follicles obtained from midline occipital scalp may be conditioned in a pro-anagenic environment for a period of up to 100 hours. The resulting cells may be enzymatically dissociated to single cells to provide in the order of 10⁵ cells. These cells may then be cultured in a suspension in a dilution of order 10⁴ cells/ml in conditions enabling neurosphere formation. The suspension culture is then filtered to harvest neurospheres only, enzyme treated to dissociate cells, and adherent monolayer expanded.

It will be understood that the invention disclosed and defined in this specification extends to all alternative combinations of two or more of the individual features mentioned. All of these different combinations constitute various alternative aspects of the invention.

### EXAMPLES

### Example 1—Harvest of Donor Tissue

Human adult skin is harvested from the occipital scalp as follows: occiput is shaved, sterilized and anaesthetized prior to harvest. A 10 mm wide, 3 cm long sample is taken at full thickness down to the fatty layer using a dual-blade scalpel, circumferentially in the axial plane around the midline.

### Example 2—Incubation of Donor Tissue with Hair Cycle Regulatory Factors

Conditioning of donor skin tissue for up to 100 hours with anti-refractory or pro-proliferative hair follicle regulatory factors increases the anagen:telogen ratio across the skin sample as well as increases inter-follicular synchrony. Pre-treatment of donor skin tissue with such factors therefore increases final cell viability, yield and uniformity. Incubation of skin is carried out within a supplemented Williams' E media environment that provides for increased in situ hair follicle viability ex vivo. Noggin or SHH as exemplar anti-refractory hair follicle cell cycle factors increase anagen:telogen ratios and therefore increase final cell viability, yield and uniformity. TGF-□2 as an exemplar pro-proliferative hair follicle cell cycle factor increases anagen:telogen ratios and therefore increase final cell viability, yield and uniformity. A combination of Noggin, SHH and TGF-□2 in supplemented Williams' E media provides for high cell viability, yield and uniformity. In practice:
1. excess subcutaneous fat is trimmed off, ensuring that dermal papilla are visible as dark puncta and retained on the fat-side of the skin sample.
2. wash remaining skin a further 3 times with 1% anti-anti in PBS
3. Sample is cut down to 4 mm×10 mm pieces by scalpel.
4. Incubation of skin pieces in supplemented Williams' E medium (containing 11.1 mM glucose and 2 mM L-glutamine) supplemented with 10 microgram/ml insulin plus: 100-500 ng/ml noggin (12-100 hours), 100-500 ng/ml SHH (12-100 hours), or 50 ng-200/ml TGF-β2 (Foitzik et al., 1999a) (12-100 hours), or combinations thereof.

### Example 3—Mechanical Processing of Skin and Digestive Enzymatic Agent Treatment

A mechanical device for skin dissociation in conjunction with enzymatic digestion for release of precursor cells from extracellular matrix can increase cell viability, yield and uniformity. An exemplar combination is the Miltenyi Biotec gentleMACS Dissociator device used in conjunction with gentleMACS enzymatic digestion kit, which together increases cell viability, yield and uniformity.

A customized approach to cell filtration outside the proprietary instructions of use provides for high cell viability, yield and uniformity.
1. Prepare gentleMACS dissociation enzyme (following volume is for 4×4 mm skin pieces).
2. Carefully mix Buffer L (435 ul) and Enzyme P (12.5 ul)
3. Carefully mix Enzyme D (50 ul) and Enzyme A (2.5 ul)
4. Add the D/A mix to the L/P mix within a C-tube
5. Place 4 skin pieces into the enzyme containing C-tubes and screw the lid on.
6. Incubate for 12 hours at 37° C.

Next Day
7. Before starting, make up at least 50 mL COLD wash media (DMEM/F12 3:1+1% anti-anti) and warm up at least 50 mL growth media (DMEM/F12 3:1 20 ng/mL EGF+40 ng/mL FGF2+2% B27)
8. Dilute the contents of each C-tube by adding 0.5 ml cold wash media
9. Tightly close C-Tube lid again and attach it upside down onto the sleeve of the gentleMACS Dissociator.
10. Select the program h_skin_01 on the gentleMACS and press start.
11. Spin the content of tube down to the bottom by 45 sec @300×G in centrifuge.
12. Empty contents from the C-tubes onto a wire mesh strainer placed over 6-well plate well(s)
13. Rinse the contents through the strainer into the well(s) using wash media
14. Removing any large obstructive pieces of tissue from the strainer as you go. Note: only remove tissue pieces after you have thoroughly washed it by pipetting cold media onto it.
15. Thoroughly rinse all C-tubes into a 50 mL tube using wash media.
16. Pipette this through the strainer into the same 6 well(s)
17. Place a 40 uM strainer on a 50 mL tube, and moisten the strainer mesh with wash media.
18. Filter the cell/media mix through the 40 uM strainer
19. Repeat the previous filtering step, by filtering the cell/media mix through a second 40 uM strainer.
20. Centrifuge cells at 300×g for 10 min. Remove supernatant completely.

### Example 4—Depletion of Potential Contaminatory Cells

Depletion of potential contaminatory cells increases cell viability, yield and uniformity compared to without such depletion. An exemplar approach uses the Miltenyi MACS magnetic cell isolation system for such live cell depletion. Using this device, depletion of terminally differentiated cell types increases cell viability, yield and consistency. One or more markers can be used for depletion of mature, non-neurosphere forming cells such as: fibroblasts (fibroblast-specific antigen1) or epithelial cells (CD45). Using this device, depletion for apoptotic cells and debris with the Miltenyi Dead Cell removal kit also increases cell viability, yield and consistency.

For depletion of apoptotic cells:
1. Per 10⁷ total cells, dilute 0.25 mL of 20× Binding Buffer Stock Solution with 4.75 mL of sterile, double distilled water.
2. Resuspend cell pellet in 100 μL of Dead Cell Removal MicroBeads per approximately 10⁷ total cells
3. Mix well and incubate for 15 minutes at room temperature (20-25° C.).
4. Choose a column type dependent on cell number and place the column in the magnetic field of the MACS Separator.
5. Prepare column by rinsing with 1× Binding Buffer (MS: 500 μL; LS: 3 mL),
6. Apply cell suspension in a suitable amount of 1× Binding Buffer onto the column (MS: 500-1000 μL; LS: 1-10 mL). Let the negative (unlabelled) cell fraction pass through into a 15 mL tube containing warmed DMEM.
7. Rinse column with appropriate amount of 1× Binding Buffer (MS: 4×500 μL; LS: 4×3 mL) and collect unlabelled cells
8. Centrifuge cells at 300×g for 10 min.
9. Prepare buffer by diluting MACS BSA Stock Solution 1:20 with autoMACS Rinsing Solution. Keep buffer cold (2-8° C.).
10. Remove supernatant from cells completely and resuspend cell pellet in 80 μL of buffer per 10⁷ total cells. Add 20 μL of MicroBeads (e.g. Anti-Fibroblast beads) per 10⁷ total cells.
11. Mix well and incubate for 30 minutes at room temperature (19-25° C.).
12. Wash cells by adding 1-2 mL of buffer per 10⁷ cells and centrifuge at 300×g for 10 minutes.
13. Aspirate supernatant completely. Resuspend up to 10⁸ cells in 500 μL of buffer.
14. Choose a column type dependant on cell number and place column it in the magnetic field of a MACS separator. Prepare column by rinsing with buffer.
15. Apply cell suspension onto the column. Collect flow-through containing unlabelled cells. Wash column with the appropriate amount of buffer. Collect unlabelled cells that pass through and combine with the effluent cell suspension.

### Example 5-3D Neurosphere Formation

1. Centrifuge unlabelled cell fraction at 350×G for 10 min.
2. Remove supernatant and resuspend pellet in 5 mL SKN growth media (DMEM/F12 (3:1), 20 ng/mL EGF, 40 ng/mL bFGF and 2% B27)
3. Removing 10 uL into a microvial and mix with 10 uL Trypan blue. Record cell count and viability.
4. Dilute the cell suspension with SKN growth media and seed into a non-tissue culture treated 6 well-plate at 1×10⁶ cells in 6 mL media per well. Record the number of wells seeded and at what precise density.
5. Incubate at 37° C. 5% CO2—this represents Culture Day 0.
6. Transfer all records and images to archive.
7. Culture Day 1: Assess for signs of contamination and dispose culture if necessary.
8. Culture Day 5 and Day 7: Photo the cells at low and high mag and measure neurosphere size. Record observations on culture growth. Proceed to 2D Monolayer Expansion if the majority of neurospheres are >50 um in diameter (approx. 1 day after first appearance). Following Day 7, monitor neurosphere size every day. Dissociation must occur prior to majority of neurospheres becoming >100 um or adherent.

### Example 6-2D Monolayer Expansion

Avoid spheres starting to adhere to the plate because they will become difficult to recover and dissociate. Target is majority ˜100 μm diameter spheres. Large irregular clusters of cells that appear in the first 1-2 days are cell aggregates, not neurospheres. They are an indication that either the cell density is too high or that the tissue was not adequately dissociated.
1. Before starting, cover a T25 flask (or however many are needed) with 0.5 μg/cm2 Laminin 511 and polymerize overnight at 4 C and warm 7-14 mL growth media (need 7 mL per T25 cells will be split into)˜DMEM/F12 3:1 with 1% pen-strep, 20 ng/mL EGF, 40 ng/mL bFGF and 2% B27, thaw 1 mL TrypLE at 4° C.
2. Take a picture of the spheres under both high and low magnification
3. Transfer all culture media containing the spheres from the culture wells into a centrifuge tube.
4. Centrifuge at 300×G for 10 mins.
5. Remove supernatant and resuspend in 1 mL TrypLE. Leave for 5 mins in the incubator
6. Take cells out of the incubator and break up spheres by pipetting up and down (with a 200 uL pipette) 100-200 times
7. Add 2 mL growth media and mix well
8. Remove 10 uL of the suspension into a small vial with 10 uL Trypan blue and cell count.
9. Record total live and dead cell counts
10. Dilute cell suspension as required in further warmed growth media
11. To prepare T25 flasks, remove Laminin 511
12. Seed at 1×10⁴ cells/cm² (=2.5×10⁵ cells in 7 mL for a T25 flask). This is P0.
13. Return cells to the incubator with minimal movement for the first 24 hours.
14. Change media for fresh growth media 24 hours later
15. Change growth medium every 3 days. Passage cells with TryPLE when flask is ˜80% confluent, re-seeding at 1×10⁴ cells/cm².

### REFERENCES

BELICCHI, M., PISATI, F., LOPA, R., PORRETTI, L., FORTUNATO, F., SIRONI, M., SCALAMOGNA, M., PARATI, E. A., BRESOLIN, N. & TORRENTE, Y. 2004. Human skin-derived stem cells migrate throughout forebrain and differentiate into astrocytes after injection into adult mouse brain. J Neurosci Res, 77, 475-86.
BIERNASKIE, J. A., MCKENZIE, I. A., TOMA, J. G. & MILLER, F. D. 2007. Isolation of skin-derived precursors (SKPs) and differentiation and enrichment of their Schwann cell progeny. Nature Protocols, 1, 2803.
FOITZIK, K., PAUS, R., DOETSCHMAN, T. & DOTTO, G. P. 1999a. The TGF-beta2 isoform is both a required and sufficient inducer of murine hair follicle morphogenesis. Dev Biol, 212, 278-89.
FUCHS, E. 2018. Skin Stem Cells in Silence, Action, and Cancer. Stem Cell Reports, 10, 1432-1438.
GE, Y., GOMEZ, N. C., ADAM, R. C., NIKOLOVA, M., YANG, H., VERMA, A., LU, C. P.-J., POLAK, L., YUAN, S., ELEMENTO, 0. & FUCHS, E. 2017. Stem Cell Lineage Infidelity Drives Wound Repair and Cancer. Cell, 169, 636-650.e14.
HWANG, I., CHOI, K. A., PARK, H. S., JEONG, H., KIM, J. O., SEOL, K. C., KWON, H. J., PARK, I. H. & HONG, S. 2016. Neural Stem Cells Restore Hair Growth Through Activation of the Hair Follicle Niche. Cell Transplant, 25, 1439-51.
JOANNIDES, A., GAUGHWIN, P., SCHWIENING, C., MAJED, H., STERLING, J., COMPSTON, A. & CHANDRAN, S. 2004. Efficient generation of neural precursors from adult human skin: astrocytes promote neurogenesis from skin-derived stem cells. The Lancet, 364, 172-178.
PANG, Z. P., YANG, N., VIERBUCHEN, T., OSTERMEIER, A., FUENTES, D. R., YANG, T. Q., CITRI, A., SEBASTIANO, V., MARRO, S., SUDHOF, T. C. & WERNIG, M. 2011. Induction of human neuronal cells by defined transcription factors. Nature, 476, 220-3.
SON, E. Y., ICHIDA, J. K., WAINGER, B. J., TOMA, J. S., RAFUSE, V. F., WOOLF, C. J. & EGGAN, K. 2011. Conversion of mouse and human fibroblasts into functional spinal motor neurons. Cell Stem Cell, 9, 205-18.
TOMA, J. G., AKHAVAN, M., FERNANDES, K. J. L., BARNABE-HEIDER, F., SADIKOT, A., KAPLAN, D. R. & MILLER, F. D. 2001. Isolation of multipotent adult stem cells from the dermis of mammalian skin. Nat Cell Biol, 3, 778-784.
TOMA, J. G., MCKENZIE, I. A., BAGLI, D. & MILLER, F. D. 2005. Isolation and characterization of multipotent skin-derived precursors from human skin. Stem Cells, 23, 727-37.
TRUONG, A., SI, E. S., DUNCAN, T. & VALENZUELA, M. 2016. Modeling neurodegenerative disorders in adult somatic cells: A critical review. Front. Biol., 11, 232-245.
TSUNEMOTO, R., LEE, S., SZUCS, A., CHUBUKOV, P., SOKOLOVA, I., BLANCHARD, J. W., EADE, K. T., BRUGGEMANN, J., WU, C., TORKAMANI, A., SANNA, P. P. & BALDWIN, K. K. 2018. Diverse reprogramming codes for neuronal identity. Nature.
VALENZUELA, M. J., DEAN, S. K., SACHDEV, P., TUCH, B. E. & SIDHU, K. S. 2008. Neural precursors from canine skin: a new direction for testing autologous cell replacement in the brain. Stem Cells Dev, 17, 1087-94.
VIERBUCHEN, T., OSTERMEIER, A., PANG, Z. P., KOKUBU, Y., SUDHOF, T. C. & WERNIG, M. 2010. Direct conversion of fibroblasts to functional neurons by defined factors. Nature, 463, 1035-1041.
YANG, H., ADAM, R. C., GE, Y., HUA, Z. L. & FUCHS, E. 2017. Epithelial-Mesenchymal Micro-niches Govern Stem Cell Lineage Choices. Cell, 169, 483-496.e13.
YU, H., FANG, D., KUMAR, S. M., LI, L., NGUYEN, T. K., ACS, G., HERLYN, M. & XU, X. 2006. Isolation of a Novel Population of Multipotent Adult Stem Cells from Human Hair Follicles. The American Journal of Pathology, 168, 1879-1888.
YU, H., KUMAR, S. M., KOSSENKOV, A. V., SHOWE, L. & XU, X. 2010. Stem Cells with Neural Crest Characteristics Derived from the Bulge Region of Cultured Human Hair Follicles. Journal of Investigative Dermatology, 130, 1227-1236.
ZHOU, D., ZHANG, Z., HE, L. M., DU, J., ZHANG, F., SUN, C. K., ZHOU, Y., WANG, X. W., LIN, G., SONG, K. M., WU, L. G. &YANG, Q. 2014. Conversion of fibroblasts to neural cells by p53 depletion. Cell Rep, 9, 2034-42.

What is claimed:

1. A method for producing a composition of neuronal precursor cells, or of cells capable of proliferation that express neural lineage biomarkers including:
treating a sample of hair follicle cells in conditions enabling the transition of hair follicle cells to a growth phase, thereby forming a sample of conditioned cells;
treating the sample of conditioned cells in conditions enabling enrichment of the number of cells containing neuronal lineage biomarkers in the sample;
thereby producing the composition of neuronal precursor or cells capable of proliferation that express neural lineage biomarkers.

2. The method of claim 1 wherein the hair follicle cells include hair follicular precursors.

3. The method of claim 2 wherein a subpopulation of hair follicular precursor cells express neuro-ectodermal biomarkers.

4. The method of any one of the preceding claims wherein hair follicle cells are provided in a sample of skin.

5. The method of any one of the preceding claims wherein the hair follicle cells are treated in conditions enabling the majority of the hair follicle precursor cells to transition to a growth phase.

6. The method of any one of the preceding claims wherein the hair follicle cells are treated with an anti-refractory hair follicle factor for promoting transition of hair follicle cells from telogen to anagen phase, thereby enabling the retention or transition of hair follicle cells in the skin to a growth phase.

7. The method of claim 6 wherein the factor is noggin or sonic hedgehog (SHH).

8. The method of any one of the preceding claims wherein the hair follicle cells are treated with a pro-growth factor for promoting transition of hair follicle cells from telogen to anagen phase, thereby enabling the retention or transition of hair follicle cells in the skin to a growth phase.

9. The method of claim 8 wherein the pro-growth factor is TGF-□2.

10. The method of any one of the preceding claims wherein the hair follicle cells are cultured in cell culture medium for hair follicle cells.

11. The method of claim 10 wherein the medium is Williams medium E.

12. The method of any one of the preceding claims wherein the skin is human skin, or wherein the hair follicles are human hair follicles.

13. The method of any one of the preceding claims wherein the skin or hair follicles are of the scalp.

14. The method of any one of the preceding claims wherein the skin or hair follicles are of the midline occipital scalp.

15. The method of any one of the preceding claims including:
treating a sample of skin, the skin including hair follicle cells, in conditions enabling the transition of hair follicle cells in the skin to a growth phase, thereby forming a sample of conditioned skin;
releasing cells from the conditioned skin into the sample;
depleting terminally differentiated cells or apoptotic cells or cell debris from the sample, thereby forming a sample of non-terminally differentiated cells; and optionally
treating the sample of non-terminally differentiated cells in conditions enabling expansion of the number of non-terminally differentiated cells in the sample;
thereby producing the composition of neuronal precursor cells or cells capable of proliferation that express neural lineage biomarkers.

16. The method of claim 15 wherein the cells are released from the skin by contacting the skin with one or more enzymes in conditions enabling degradation of the extracellular matrix of the conditioned skin for release of the cells into the sample.

17. The method of claim 16 wherein the enzyme is selected from the group consisting of: trypsin, Dnase, dispase, and collagenase.

18. The method of claim 15 wherein the cells are released from the conditioned skin by mechanically disrupting the extracellular matrix of the conditioned skin for release of the cells into the sample.

19. The method of claim 18 wherein the mechanically disruption includes manual trituration.

20. The method of claim 15 including the step of removing non cellular components from the sample after release of cells from the conditioned skin into the sample and prior to depletion of terminally differentiated cells from the sample.

21. The method of claim 15 wherein the terminally differentiated cells are depleted from the sample by contacting the sample with a reagent for selectively depleting terminally differentiated cells from the sample in conditions enabling selective depletion of terminally differentiated cells from the sample.

22. The method of claim 21 wherein the agent is an antibody that binds to terminally differentiated cells but not to non-terminally differentiated cells.

23. The method of claim 22 wherein the agent is an antibody that does not bind to neural precursor cells.

24. The method of claim 23 wherein the antibody binds to cells of the epidermis or dermis.

25. The method of claim 24 wherein the antibody binds to epithelial cells or keratinocytes, or to fibroblasts.

26. The method of claim 22 wherein the antibody binds to fibroblast-specific antigen 1 or to CD45.

27. The method of any one of the preceding claims wherein at the completion of the depletion step, the sample contains no more than about 5% by number of terminally differentiated cells.

28. The method of any one of claims 1 to 14 including:
treating a sample of hair follicle cells in conditions enabling the transition of hair follicle cells to a growth phase, thereby forming a sample of conditioned cells;
providing conditions to the sample of conditioned cells to enable dissociation of cells into single cells;
treating the sample of conditioned cells in conditions enabling the formation of a sample of neurospheres from the cells of the sample of conditioned cells;
providing conditions to the sample of neurospheres to expand the number of cells of the neurospheres;
thereby producing the composition of neuronal precursor cells or cells capable of proliferation that express neural lineage biomarkers.

29. The method of any one of the preceding claims wherein the composition produced by the method has the following characteristics:
<5% of cells express astroglial GFAP, adiponectin, oligodendrocyte 04, myofibroblast SMA.
>90% of cells are positive for nestin, CD133 and 13111-tubulin.

30. The method of any one of the preceding claims wherein the composition produced by the method has a neuronal yield of 90 to 100% following in vitro neuronal differentiation.

31. The method of any one of the preceding claims wherein the cells of the composition are expandable to produce in the order of 10⁷ homogenous and unipotent neuronal precursor cells within 4 weeks.
